SEQUENCE LISTING (11 sequences)

SEQ ID NO: 1 - DNA, 22 bp, Artificial
  Sanger sequencing primer
gtatgggattttgctaaacaac

SEQ ID NO: 2 - protein, 12 aa, Artificial
  phage dispaly isolated peptide
TSITPWFFLSRP

SEQ ID NO: 3 - protein, 12 aa, Artificial
  phage display isolated peptide
LPISNPLPIKRS

SEQ ID NO: 4 - protein, 12 aa, Artificial
  phage display isolated peptide
TGRHTPWPHFMP

SEQ ID NO: 5 - protein, 12 aa, Artificial
  scrambled negative control peptide
SLFWTTPIPSFR

SEQ ID NO: 6 - protein, 7 aa, Artificial
  fragment of phage display isolated peptide
  MISC_FEATURE (6)..(6): X represents any amino acid
PWFVLXR

SEQ ID NO: 7 - protein, 5 aa, Artificial
  fragment of phage display isolated protein
PWFLV

SEQ ID NO: 8 - protein, 12 aa, Artificial
  isolated phage display peptide
AATTPWFVLSRA

SEQ ID NO: 9 - protein, 12 aa, Artificial
  phage display isolated peptide
TAAAPWFVLARP

SEQ ID NO: 10 - protein, 7 aa, Artificial
  fragment of phage display isolated peptide
PWFVLSR

SEQ ID NO: 11 - protein, 7 aa, Artificial
  fragment of phage display isolated peptide
PWFVLAR

(19) United States
(12) Patent Application Publication
(10) Pub. No.: US 2007/0066801 A1
(43) Pub. Date: Mar. 22, 2007

(54) Identification of reagents for the diagnosis and study of francisella

(76) Inventors: Jeffrey A. Engler, Birmingham, AL (US); Vincent E. Escuyer, Vestavia Hills, AL (US)

Correspondence Address: BRADLEY ARANT ROSE & WHITE, LLP;INTELLECTUAL PROPERTY DEPARTMENT-NWJ, 1819 FIFTH AVENUE NORTH, BIRMINGHAM, AL, 35203-2104, US

(21) Appl. No.: 10/564,950

(22) PCT Filed: Jul. 14, 2004
(86) PCT No.: PCT/US04/22460

§ 371 Date: Jul. 24, 2006

Related U.S. Application Data
(60) Provisional application No. 60/487,530, filed on Jul. 14, 2003.

(51) Int. Cl. C07K 14/195; C07K 16/12; C12Q 1/68; C07H 21/04; C12P 21/06; C12N 15/74

(52) U.S. Cl. 530/350; 530/388.4; 435/6; 435/69.1; 435/252.3; 435/471; 536/23.7

(57) ABSTRACT

Novel reagents capable of identifying, or aiding in the identification of, Francisella strains, such as, but not limited to, those identified in playing a role in human diseases or those identified as capable of being used as a biological weapon, are disclosed. In addition, methods for the identification and isolation of the reagents are disclosed. In one embodiment the reagents are peptide sequences. The reagents may be used in diagnostic assays and kits to diagnose the presence of such Francisella strains. In addition the agents identified may be used as research tools for the study and characterization of such Francisella strains.

### FIELD

The present disclosure relates to the analysis of bacteria. Specifically, the current disclosure relates to reagents that are capable of identifying bacteria causing human disease and/or posing a biological threat.

### BACKGROUND

The growing concern for the use of biological agents as weapons in terrorist incidents has prompted Federal health agencies to accelerate measures to protect the public from such threats. In February, 2002, the National Institute of Allergy and Infectious Diseases (NIAID) released its biodefense research agenda for CDC category A agents. The agenda described the high priority goals for immediate, intermediate and long-term research related to counter bioterrorism (1).

Francisella tularensis, the agent causing tularemia, is a CDC category A agent and is considered as a major biological threat. F. tulareisis has long been considered as a potential biological weapon. In the 1950's and the 1960's, the US military developed weapons that would disseminate F. tularensis aerosols and by the late 1960s, F. tularensis was one of several biological weapons stockpiled (2). A parallel effort by Soviet Union continued into the early 1990s and resulted in weapons production of F. tularensis strains engineered to be resistant to antibiotics and vaccines (3). F. tularensis is a good candidate for a biological weapon due to its very high infectivity. The infectious dose can be as low as 10 to 50 microorganisms if inhaled (4). In 1969, a World Health Organization expert committee estimated that an aerosol dispersal of 50 kg of virulent F. tularensis over a metropolitan area with 5 million inhabitants would result in 250,000 incapacitated casualties, including 19,000 deaths (5). Recently, the Centers for Disease Control and Prevention examined the expected economic impact of bioterrorist attacks and estimated that the total base cost to society of an F. tularensis aerosol attack would be $5.4 billion for every 100,000 persons exposed (6).

Tularemia is a zoonotic disease caused by F. tularensis and occurs mainly in the Northern Hemisphere (reviewed in 7). In the United States, the disease is most prevalent in Arkansas, Illinois, Missouri, Texas, Virginia and Tennessee (8). Tularemia agent is primarily recovered from lagomorphs (rabbits), rodents and arthropods (ticks and deer flies) within the United States and from water, mosquitoes and aquatic mammals outside the United States. The rabbit is the vertebrate most commonly associated with tularemia in North America. The reported incidence of tularemia in the United States since 1967 has been fewer than 200 cases per year (9). However, in the event of an intentional release, a large number of cases in a local geographical area would be expected.

Tularemia is acquired under natural conditions by direct inoculation (such as, but not limited to, arthropod bite), animal contact such as, but not limited to, skinning or eating infected animals, by drinking contaminated water, or via the airborne route (10-12). As few as 10 to 50 microorganisms given by aerosol can cause infection in humans (4). To date, tularemia transmission from patient to patient has never been reported.

Tularemia can be divided into the ulceroglandular (˜75% of the patients) and the typhoidal (˜25% of the patients) forms, based on the clinical symptoms. The average incubation period is 3 to 6 days. Patients with the ulceroglandular form of the disease develop nonspecific symptoms consisting of fever, chill, headache cough and myalgia (13). A cutaneous ulcer occurs, associated with large peripheral lymph nodes (>1 cm) in 60% of patients and is the most common sign of tularemia. The typhoidal form presents symptoms very similar to the ulceroglandular form, but lymph nodes are usually smaller (<1 cm) and skin lesions are generally absent. Approximately 80% of the patients with the typhoidal form also contract pneumonia; this linkage probably accounts for the higher mortality associated with this form of the disease (30-60% if untreated) (8). As a result of its higher mortality, the typhoidal form would be the most likely form in case of an act of bioterrorism.

A live attenuated vaccine has been developed from the avirulent live vaccine strain (LVS) initially used by the Russian in the 1930's (14, 15). It is used to protect people routinely working with F. tularensis. This vaccine has been shown to protect human volunteers against an aerosol challenge with virulent F. tularensis (16). Recent investigations of mouse models have suggested that the delivery site may influence the host defense mechanisms employed to combat the infection (17). The LVS is currently available as an Investigational New Drug and is under review by the US Food and Drug Administration.

Rapid diagnostic testing for tularemia is not widely available (reviewed in 41, 42). The organism can be isolated from blood, sputum, skin, or mucosal membrane lesions of an affected individual, but microbiologic diagnosis can be difficult due to unusual growth requirements for Francisella strains and/or due to the overgrowth of commensal bacteria. Diagnosis of primary typhoidal tularemia is also difficult because signs and symptoms are nonspecific and frequently there is no suggestive exposure history (43). Because of its infrequent occurrence, its symptoms might be misidentified, resulting in a delay in starting effective antibiotic treatment. In the case of an intentional or accidental release of aerosolized Francisella strains, such as, but not limited to, F. tularensis, it may be the clustered occurrence and unusual epidemiologic features that alert authorities to the intentional release. A number of assays may be used to identify Francisella strains, such as, but not limited to, F. tulareisis, but the availability of such assays (for example, antigen detection, PCR, ELISA; compared in (44)) may only be available in research and reference laboratories. Serologic assays to detect the presence of specific antibody may not be diagnostic for many days after infection and would hinder effective treatment and/or containment of an intentional release. Any diagnostic reagent for strains of Francisella would be a major benefit, since the treatment for tularemia is the same for all strains (62-64). In cases of geographically concentrated outbreaks of tularemia (such as, but not limited to in a biological attack, an inadvertent release or a naturally occurring outbreak), the inability to rapidly diagnose the causative Francisella agent could be the key impediment to an effective public health response.

Therefore, a rapid and sensitive diagnostic assay is needed to allow a rapid response in the event of an intentional or accidental release of pathogenic Francisella strains. Such a diagnostic assay is currently lacking in the art.

### BRIEF DESCRIPTION OF THE FIGURES

FIG. 1 shows a representative diagram of the Negative/Positive biopanning procedure used in the present disclosure. In this example, peptide sequences that bind to F. tulareisis LVS and not to F. philomiragia were isolated using both negative and positive selection steps as indicated (59). Phage that did not bind F. philomiragia was applied to F. tulareisis LVS and the bound phage were eluted with low pH buffer. The eluted phages were then amplified in additional rounds of selection.

FIG. 2 shows relative phage binding to F. tulareisis LVS, F. novicida and F. philomiragia. 4-4-2 phage (expressing peptides having the sequence of TSITPWFFLSRP) (SEQ ID NO: 2) and OV1 phage (negative control, expressing peptides having the sequence of TGRHTPWPHFMP) (SEQ ID NO: 4) were used. Identical aliquots of phage were allowed to bind for 2 hours at each temperature shown. The columns show the ratio of binding to F. tulareisis LVS and F. novicida, compared to F. philomiragia (ratio=1). The ratios observed did not significantly change when the amount of phage added was varied from 0.1 to 100 phage added per bacterium (data not shown).

FIG. 3 shows phage binding to F. tularensis LVS (FI), F. novicida FN), F. philomiragia (FP), S. aureus (SA), E. coli (EC), and E. hirae (ENT) at 4° C. and at 37° C. 4-4-2=4-4-2 phage expressing peptides having the sequence of TSITPWFFLSRP) (SEQ ID NO: 2); negative=OV1 phage (expressing peptides having the sequence TGRHTPWPHFMP) (SEQ ID NO: 4).

FIG. 4 shows Trypsin sensitivity of phage binding to F. tulareisis LVS CFM and to F. philomiragia (FP) at 37° C. 4-4-2=4-4-2 phage expressing peptides having the sequence of TSITPWFFLSRP) (SEQ ID NO: 2); negative=OV1 phage (expressing peptides having the sequence TGRHTPWPHFMP) (SEQ ID NO: 4).

FIG. 5 shows the specificity of phage binding to F. tulareisis LVS and F. novicida. 1×10¹¹ bacteriophage were mixed with peptide p442 or p42S at the concentration shown and allowed to bind to 1×10⁹ F. tulareisis LVS or 1×10⁹ F. novicida bacteria for 1 hour at 4° C. in 1× PBS. After binding, bacteria were washed three times with ice cold 1×PBS. Bound phage were eluted with 0.2 M glycine buffer (pH 2.2) and titered on E. coli strain ER2738. Phage used for binding were 4-4-2 (expressing peptides having the sequence of TSITPWFFLSRP) (SEQ ID NO: 2) or OV1 (expressing peptides having the sequence TGRHTPWPHFMP) (SEQ ID NO: 4) which served as a negative control. p442=competing peptide with sequence TSITPWFFLSRP (SEQ ID NO: 2); p442S=non-competing peptide with the sequence SLYWTTPIPSFR (SEQ ID NO: 5).

FIG. 6 shows the effect of buffer composition on phage binding to F. tulareisis LVS (FC), F. novicida (FN) and F. philomiragia (FP). 10¹¹ bacteriophage were added to 10⁹ bacteria in 0.2 ml of the Tris-Mg buffer, 1×PBS and 0.1×PBS as indicated. After 1 hour at 4° C., the bacteria were washed 3 times with the same buffer used in the binding reaction (4° C.). Bound bacteriophage were eluted with 0.2 M glycine (pH 2.2) and titered on E. coil strain ER2738. Phage used for binding were 4-4-2 (expressing peptides having the sequence of TSITPWFFLSRP) (SEQ ID NO: 2) or OV1 (expressing peptides having the sequence TGRHTPWPHFMP) (SEQ ID NO: 4) which served as a negative control.

FIG. 7 shows the effect of growth media on phage binding to F. tulareisis LVS (FT), F. novicida (FN) and F. philomiragia (FP). Bacteria were grown in either trypticase soy (TSB) or Chamberlain minimal medium to mid-log phase. Bacteria were collected by centrifugation and resuspended in 1×PBS at a concentration of 5×10⁹ bacteria/ml. 10¹¹ bacteriophage were added to the bacteria in 0.2 ml of 1×PBS. After 1 hour at 4° C., the bacteria were washed 3 times with 1×PBS (4° C.). Bound bacteriophage were eluted with 0.2 M glycine (pH 2.2) and titered on E. coli strain ER2738. Phage used for binding were 4-4-2 (expressing peptides having the sequence of TSITPWFFLSRP) (SEQ ID NO: 2) or OV1 (expressing peptides having the sequence TGRHTPWPHFMP) (SEQ ID NO: 4) which served as a negative control.

### DETAILED DESCRIPTION

Study of the biology and the mechanisms of pathogenicity of Francisella have been impaired by a lack of research and diagnostic tools. This lack of information has hampered the development of diagnostic assays and kits, as well as the identification of novel proteins and other targets for use in drug discovery programs and in vaccines. Therefore, it would be advantageous to provide reagents that identify Francisella strains, such as, but not limited to, those identified in playing a role in human diseases or those identified as capable of being used as a biological weapon. In one embodiment peptide sequences are identified as such reagents. Furthermore, it would be advantageous to provide diagnostic assays using the reagents identified for the identification of Francisella strains, such as, but not limited to, -those identified in playing a role in human diseases or those identified as capable of being used as a biological weapon. Further still, it would be advantageous to provide diagnostic assays suitable for the rapid detection of such Francisella strains. Such rapid detection may prove useful, for example, in the event of an intentional or accidental release of Francisella strains. Finally, it would be advantageous to provide such reagents as research tools for the study and characterization and to provide vaccines useful for protection against Francisella strains those identified in playing a role in human diseases or those identified as capable of being used as a biological weapon.

Francisella strains, such as F. tularensis, are tiny, non-motile, aerobic, gram-negative coccobacillus. These strains are fastidious organism and usually requires cysteine supplementation for good growth on general laboratory media. F. tulareisis has a thin capsule consisting of lipids, carbohydrates and proteins, all of which may contribute to the virulence (18). Biochemically, the organism is fairly inert, using glucose as the carbon source. It is a hardy non-spore forming microorganism, that is able to survive for weeks at low temperatures in water, moist soil, hay and decaying animal carcasses (19). F. tulareisis has been divided into two major biovars (20). F. tulareisis biovar tularensis (type A) is the most common in the United States and is recovered from rodent and ticks and is highly virulent for rabbits and human. F. tularensis biovar paleartica (type B) is more common outside the United States and is recovered from water, mosquitoes, and aquatic mammals, and is relatively avirulent for rabbits and human. Both subspecies cannot be distinguished serologically although they may be differentiated by 16S RNA sequencing (21). Other Francisella strains include F. philomiragia (which shares a moderate 30-40% homology to F. tulareisis) and F. novicida (which shares an approximate 90% homology to F. tularensis) (22).

Very little is known about the mechanisms of virulence of Francisella. This pathogen is capable of growing in macrophages from rodents including mice, rats and guinea-pigs as well as human monocytes (23, 24). Francisella appears to be able to inhibit phagosome-lysosome fusion (23). However, the intracellular compartment occupied by Francisella is acidified, an event thought to facilitate acquisition of iron, which is essential for growth (25). There is also evidence that Francisella can replicate in some types of phagocytic cells, such as, but not limited to, fibroblasts and hepatocytes (26, 27). Data from experimental animal infections suggest that in vivo, F. tulareisis is rarely found extracellularly and replicates predominantly within the macrophage (24, 25).

Molecular mechanisms involved in the virulence and the intracellular growth of Francisella are not known. Two genes required for the intramacrophage growth of Francisella novicida, a closely related bacterium, have been reported (28), but their exact function remains to be identified. There have been a number of reports of components in the outer membrane and LPS and their potential role(s) in inducing an immune response (29-36), but the potential use of these components as therapeutic targets or as diagnostic reagents has not been described. At least one cell-surface polysaccharide antigen (O-antigen) has been isolated by acid hydrolysis of the bacterial lipopolysaccharide (37; 38); this antigen is related to antigens on other bacteria such as, but not limited to, Pseudomonas aeruginosa and Shigella dysenteriae and probably would not provide an appropriate target for therapeutic or diagnostic reagents. Further, the O-antigen by itself appears not to confer immunity to systemic or aerosol challenge with F. tularensis (39).

In this disclosure, new methods for the identification and isolation of novel reagents capable of identifying, or aiding in the identification of, Francisella strains, such as, but not limited to, those identified in playing a role in human diseases or those identified as capable of being used as a biological weapon, are disclosed. The reagents may be used in diagnostic assays and kits to diagnose the presence of such Francisella strains in a sample suspected of containing a Francisella strain. The sample may be obtained from a subject, such as a human or an animal, or may be obtained from an environmental source. In addition the reagents identified may be used as research tools for the study and characterization of Francisella strains. In one embodiment, the reagents identified are peptides. In one embodiment, the reagents identified may be used to identify molecular targets and these molecular targets may be used as antigens to provide vaccines useful for protection against Francisella strains, such as, but not limited those identified in playing a role in human diseases or those identified as capable of being used as a biological weapon.

The peptides or other reagents identified by the methods disclosed herein, such as those peptides having the amino acid sequences set forth in SEQ ID NOS. 2, 3, 6, 7, 8, 9, 10, and 11, may be used in kits to determine the presence of a Francisella strain in a sample. Such diagnostic kits are well known in the art and will generally be prepared so as to be suitable for determining the presence o of a Francisella strain that will bind to the peptides or other reagents identified by the methods disclosed. These diagnostic kits will generally include the reagent, such one or more peptides having the amino acid sequences set forth in SEQ ID NOS. 2, 3, 6, 7, 8, 9, 10, and 11, along with suitable means for detecting the reagents as would be readily understood by one skilled in this art. For example, the means for detecting binding of the reagent to the Francisella stain may comprise a detectable label that is linked to the reagent In one embodiment where the reagent is a peptide, the peptide may be linked to the detectable label. Non-limiting examples of detectable labels may include an enzyme, a chromophore, a flourophore, an affinity tag, an additional peptide sequence or a. radioligand. The detectable label may be detected directly or indirectly. Methods for the detection of such detectable labels are well known in the art. Furthermore, one or more detectable labels may be used in combination or in series.

Such kits can then be used in diagnostic methods to detect the presence of a Francisella strain wherein one obtains a sample suspected of having such a Francisella strain, such as a sample taken from a subject. The sample may be taken from the subjects, blood, saliva, urine, cerebrospinal fluid, genitourinary tract, tissues, bone, muscle, cartilage, sputum, a mucosal membrane or skin. Reagents are introduced to the sample and allowed to bind to any Francisella strain that may be present in the sample, and the reagents are detected. A positive detection would indicate the presence of a Francisella strain.

Such kits may also contain a positive control Francisella strain, a negative control bacterial strain, and negative control reagents. In the case where the reagents are peptides, the negative controls may be peptides that have a sequence divergent from the sequence of the peptides (such as the peptide disclosed in SEQ ID NO. 4), or that have the same amino acid composition as the peptides, but in a scrambled sequence (such as the peptide disclosed in SEQ ID NO. 5)

One means for identifying such reagents is the technique of phage display. In one method of phage display, random peptide libraries are displayed on the surface of bacteriophage M13 (44-47). Such libraries have been used in a number of different applications, including epitope mapping (47-50), mapping protein-protein contacts (47), and identification of peptide mimics of non-peptide ligands (51-54). Phage display methods do not depend on the protein target being immunogenic, an advantage over the use of monoclonal antibodies for developing diagnostic reagents. Bioactive peptides can be selected either against immobilized purified targets (55) or against intact cells (56-59). One such library is the Ph.D.-12 library (New England Biolabs), in which 2.7×10⁹ different random 12-amino-acid peptides have been fused to the amino terminus of the pIII protein, resulting in 3 to 5 copies of a random peptide sequence on the surface of each phage. Other phage display libraries may also be used, as well as other types of libraries.

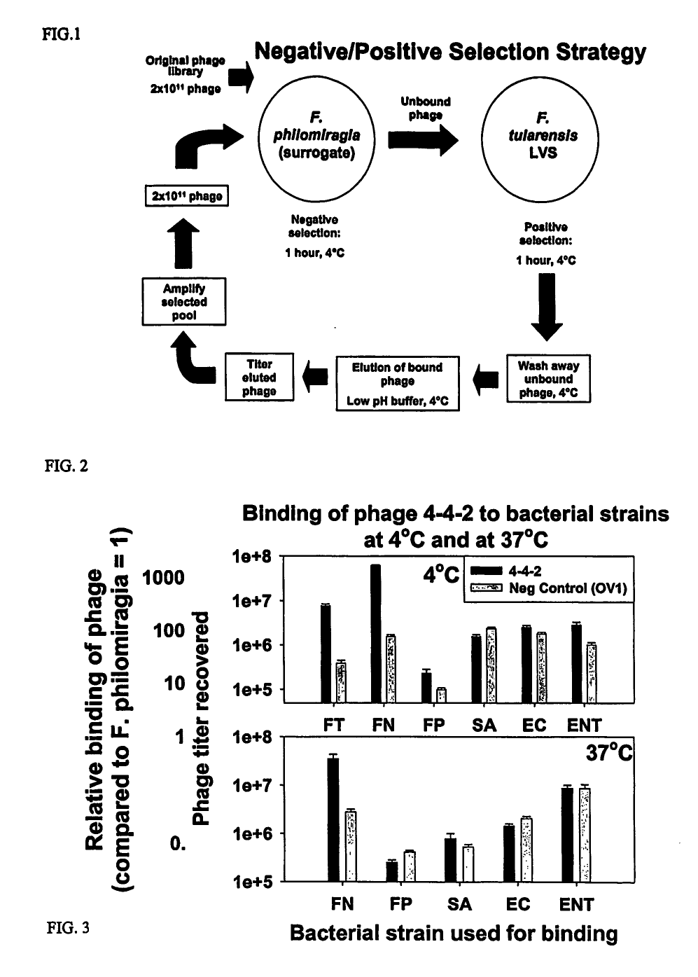

In the present disclosure, the standard phage display biopanning strategy has been modified to allow for selection of peptides that are capable of binding to Francisella strains. The Francisella strain to be identified can vary depending on the selection of organisms in the Negative/Positive selection strategy described herein. In one embodiment, the target Francisella strain is selected from the group consisting of F. tularensis and F. novicida, or sub-species thereof. This strategy has been termed “Negative/Positive” selection, because the first step in each round of selection is a negative selection step, to remove phages that bind to related cell types or organisms. This negative selection is followed by a positive selection that favors binding to the target cell type or organism. A schematic of the steps involved in one embodiment of the Negative/Positive selection step is shown in FIG. 1. The details of the Negative/Positive selection are discussed below.

In one embodiment, the Negative/Positive selection strategy illustrated in FIG. 1 was used to identify peptides that bind to F. tularensis LVS and not to F. philomiragia. In this embodiment, F. philomiragia is used in the negative selection step and is termed the “surrogate strain” and F. tularensis LVS is used in the positive selection step and is termed the “target strain”. F. tularensis LVS and F. philomiragia share about 30-40% DNA homology (22). Therefore, some of the differences should represent differences in targets found on the cell surface. The Negative/Positive selection strategy could also detect differences in the amount of individual targets (such as, but not limited to, protein targets) on the surface of the surrogate and target strains, provided that this target was in substantially higher abundance on the target strain than on the surrogate strain. It should be noted that organisms other than F. tularensis LVS and F. philomiragia can be used as the target and surrogate strains in the Negative/Positive biopanning procedure. For example, F. tularensis Shu-4 could be used in place of F. tularensis LVS and F. novicida could be used in place of F. philomiragia. These substitutions are exemplary only and others may be used. In one embodiment the substituted Francisella strains are from the same biovar. In an alternate embodiment, the substituted Francisella strains are from a different biovar. In addition, more than one surrogate strain or target strain could be used. For example, both F. novicida and F. philomiragia could be used in the negative selection step. By altering the organisms used in the negative selection steps, reagents specific for recognition of desired Francisella strains and/or reagents specific for only the human pathogenic forms of Francisella. Such reagents can be used as important diagnostic tools. The availability of any diagnostic tool for Francisella strains will also reduce the chances for confusion of tularemia infection with other diseases with similar symptoms (11, 63).

The percent homology between the Francisella strain used in the negative selection step (the surrogate strain) and the Francisella strain used in the positive selection step (the target strain) may impact the nature of the reagents identified that bind to the target Francisella strain. For example, if the surrogate strain shares a low homology with the target strain, a wider variety of reagents will be identified. Conversely, if the surrogate strain shares a high homology to the target strain, a restricted set of reagents will be identified. The homology may be defined in terms of nucleic acid sequence. In one embodiment, the target strain shares 30-40% homology in its DNA chromosome to the surrogate strain. In an alternate embodiment, the target strain shares 40%-75% homology in its DNA chromosome to the surrogate strain. In yet an alternate embodiment, the target strain shares a 75%-99% homology in its DNA chromosome to the surrogate strain. The percent homology of the target strain to the surrogate strain can be selected by one of skill in the art. Furthermore, the percent homology between the target and surrogate strains can be modulated to identify reagents with desired characteristics.

In addition, the percent homology of the target strain to known human pathogenic forms of the Francisella strains can impact the reagents identified. The closer the homology between the target strain and the known human pathogenic forms of the Francisella strains, the more likely the reagents identified in the selection strategy will react with pathogenic Francisella strains. In one embodiment, the target strain is related to human pathogenic Francisella strains such that the reagents identified with the target strain will react with a number of pathogenic Francisella strains. Such an increased capacity for recognizing numerous Francisella strains can be of value, especially if new pathogenic forms of Francisella are required to be detected, as may be the case if Francisella were used as a biological weapon. This aspect is applicable to diagnostic testing and treatment, since the treatment for all Francisella infections is identical. In one embodiment, the target strain is selected with a percent homology to known human pathogenic Francisella strains, such as, but not limited to, F. tularensis Shu-4, such that the reagents binding the target strain will react with the human pathogenic forms of Francisella. In one embodiment, the target strain shares 30-40% homology in its DNA chromosome to a human pathogenic forms of Francisella. The percent homology may be determined at the nucleic acid level. In an alternate embodiment, the target strain shares 40-75% homology in its DNA chromosome to a human pathogenic forms of Francisella. In a yet another alternate embodiment, the target strain shares 75-95% homology in its DNA chromosome to a human pathogenic forms of Francisella. The percent homology of the target strain to the human pathogenic forms of F. Tularensis can be selected by one of skill in the art. Furthermore, the percent homology of the target strain to the human pathogenic forms of F. tularensis can be modulated to identify reagents with desired characteristics.

As reagents with specificity for human pathogenic forms of Francisella are identified, the reagents can be used to identify the cell-surface molecule on the pathogenic strains and to identify drug targets specific for the human pathogenic strains.

The affinity and/or avidity of the reagents selected may also be impacted. For example, if the surrogate strain shares low homology with the target strain, then the reagents selected may be of decreased affinity and/or avidity. The number of selection rounds may also impact the affinity and/or avidity of the reagents identified as discussed below.

In summary, by choosing the homology of the surrogate and target strains used in the negative and positive selection steps, the characteristics and specificity of the reagents identified can be modulated. Given the teachings of the present disclosure, one of ordinary skill in the art would be able to choose appropriate surrogate strains and target strains without undue experimentation.

As used in this disclosure, the term “percent homology” of two nucleic acid sequences is determined using the DNA:DNA hybridization method described in reference 22. Other methods of determining the percent homology may be used as is known in the art.

As discussed above, the 12 amino acid Ph.D.-12 Phage Display library was used in one embodiment of the Negative/Positive selection method described herein. Other phage display libraries or other peptide libraries could be used and should be considered within the scope of the present disclosure. In the remainder of this specification, specific reference is made to an embodiment of the methods where peptide sequences are identified as the reagents. In one embodiment of the method disclosed, F. philomiragia was used as the surrogate strain and F. Tularensis LVS was used as the target strains. This embodiment is exemplary only and serves to illustrate the operation of the methods disclosed, and can be altered as discussed above. F. Philomiragia bacteria were grown in trypticase-soybean broth (supplemented with 0.1% cysteine) to approximately 10⁹ (growing in mid-exponential phase). Bacteria were washed at least once with 1×PBS (4° C.). 2×10¹¹ phage particles were added to approximately 10⁹ F. philomiragia bacteria in 4° C. 1×PBS and allowed to bind for 1 hour at 4° C. In alternate embodiments, the binding step can occur at other temperatures, such as, but not limited to, 37° C. In these cases, the wash steps performed both before and after the binding step are performed at the temperature of the binding step. Francisella are resistant to cold, so the 4° C. binding step does not affect their viability. After one hour incubation, F. philomiragia bacteria were collected by centrigation and the supernatant added to approximately 10⁹ F. tularensis LVS bacteria in 4° C. 1×PBS. F. Tularensis LVS bacteria were grown as described above for F. philomiragia, and prepared for phage binding as described above. After one hour incubation in 1× PBS at 4° C., F. tularensis LVS bacteria were washed three times with 1×PBS (4° C.) and then bound phages eluted by treatment with low pH buffer (0.2 M glycine, pH 2.2, at the same temperature used for the binding steps) and neutralized with 1 M Tris/HCl, pH 9.1. This step partially inactivates the bacteria themselves. Any remaining viable Francisella bacteria are removed by filtration through a 0.22 micron filter (without detectable loss of phage). Eluted phages were amplified in 20 mL Luria-Bertani medium containing the E. coli ER2783 at 37° C. (other appropriate E. coli strains may also be used). Phage from liquid cultures are obtained by clearing the supernatant twice by centrifugation at 12,000 g for 15 min (each at 4° C.), and precipitating with ⅙ vol. of 20% poly(ethylene glycol)-8000/2.5 m NaCl at 4° C. overnight. Phage pellets are re-suspended in 1 ml NaCl/Tris (50 mm Tris/HCl, 150 mm NaCl), and precipitated with 20% poly(ethylene glycol)-8000/2.5 m NaCl for 1 hr. Amplified phage are resuspended in 200 ml NaCl/Tris, 0.02% NaN₃, and are used for the subsequent round of biopanning (if desired) to select appropriate peptides.

In the embodiment discussed above, three additional rounds of selection were completed to select for peptides that bound F. tularensis LVS preferentially as compared to F. philomiragia. Increasing the rounds of selection may decrease the number of candidate phage peptide sequences for analysis and select and/or enrich for those phage having higher specificities and/or higher affinities for F. tularensis LVS binding. However, one round of selection may be sufficient. In other instances, additional rounds of selection may be used. One of ordinary skill in the art will be able to determine if additional rounds of selection are required to isolate reagents with desired characteristics. For example, if only reagents with the highest binding affinity are desired, multiple rounds of selection may yield superior results. After 4 rounds of selection using the biopanning strategy above, individual plaques were amplified and phage DNA purified for DNA sequencing, using a modified Sanger sequencing reaction and the sequencing primer: 5′ GTATGGGATTTTGCTAAACAAC 3′ (SEQ ID NO: 1). From the DNA sequence, the amino acid sequence of the peptide was determined. Two 12mer peptide sequences were isolated after 4 rounds of selection: TSITPWFFLSRP (SEQ ID NO: 2) (phage plaque 4-4-2: found in 4 out of 9 plaques sequenced) and LPISNPLPIKRS (SEQ ID NO: 3) (2 out of 9 plaques sequenced). After 7 rounds of selection, each of these phages were still present in the selected population.

The binding and elution conditions can be selected to favor the isolation of peptides with desired characteristics. These techniques are well known to those of ordinary skill in the art. As an example of such methods, to favor the isolation of phages that express sequences that bind tightly to the target strain, the incubation times for the binding and elution steps can be varied. In one embodiment, to favor the isolation of phages that express sequences that bind tightly to the target strain an initial short (5-10 minutes) elution with 0.2 M glycine (pH 2.2) is used, followed by a second longer elution with the same buffer. Phage that express sequences that do not bind tightly to the target strain will be eluted during the first elution, with phage that express sequence that bind tightly to the target strain being eluted during the second elution. In addition, the first elution could be performed with glycine buffer with a different pH. In one embodiment, the pH of the glycine buffer used in the first elution is pH 3.0 to 6.0. The above example is exemplary only and not meant to restrict other methodologies used to select phages expressing sequences with desired binding characteristics.

In addition to the amino acid sequences disclosed herein in SEQ ]ID NOS. 2-11, the present disclosure also includes fragments and derivatives of the amino acid sequences disclosed in SEQ IS NOS. 2-11. The fragments are derived from their parent peptides and comprise at least three contiguous amino acids. In an alternate embodiment, the fragments comprise at least 5 contiguous amino acids. A derivative is defined as an amino acid sequence having conservative amino acid substitutions as compared to the parent sequence. Conservative amino acid substitutions may be viewed as substitutions may be any substitution of amino acids within the same group as shown in Table 1.1 of Biochemistry (Geoffrey Zubay, coordinating author, second edition)

The terms “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Identity or similarity is evaluated using any of the variety of sequence comparison algorithms and programs known in the art. Such algorithms and programs include, but are by no means limited to, TBLASTN, BLASTP, FASTA, TFASTA, CLUSTALW, FASTDB

As an alternative to the use of phage display, single-chain antibody libraries (72) can be screened using the “Negative/Positive” selection strategy described. Selection could be done and the selected phage sequences analyzed using approaches like those described elsewhere in this disclosure. In addition, one of the Applicants has constructed a phage display single chain antibody library, taking advantage of the natural single chain antibodies found in camels and llamas. These libraries contain variable regions of 14-19 amino acids and are expressed on the surface of M13 bacteriophage grown in E. coli. The “Negative/Positive” selection strategy could be used for these selections as well.

The phage isolated carrying the TSITPWFFLSRP (SEQ ID NO: 2) 12 mer peptide showed preferential binding to F. tularensis LVS and F. novicida as compared to F. philomiragia (FIG. 2). 10¹¹ phages were added to cultures of 10⁹ F. tularensis LVS, F. novlcida, or F. Philomiragia cells (mid-log phase growth) in 1×PBS and incubated at 4° C. or at 37° C. for two hours. After incubation, the cells were washed three times with 1×PBS at room temperature Bound bacteriophage were eluted with 0.2M glycine (pH 2.2) and dilutions were plated onto E. coli ER2783 cells as described. Phage titers were in the range of 1×10⁵ to 3×10⁸ plaques recovered. In FIG. 2, the ratio of binding of the phages to F. tularensis LVS and F. novicida to F. Philomiragia were compared. At both binding temperatures, there was greater than a 100 fold binding of phage 4-4-2 expressing peptides having the sequence of SEQ ID NO: 2 to F. Tularensis LVS and F. novicida as compared to F. philomiragia. As a negative control, a phage expressing the sequence TGRHTPWPHFMP (OV1) (SEQ ID NO: 4), which was obtained by selection on ovarian cancer cells was used. The OV1 phage showed approximately equal binding to iall strains tested. Phage binding tests of the peptide expressing SEQ ID NO: 3 showed no specific binding to F. tularensis LVS compared to F. philomiragia.

SEQ ID NO: 2 was tested for binding to other bacteria that might be encountered in a clinical laboratory (FIG. 3). The binding of phage expressing peptides having the sequence of SEQ ID NO: 2 to gram-negative E. coli DH5α (a strain lacking sex pili and unable to grow M13 phage), gram positive Staphylococcus aureus (ATCC 6538), and gram positive Enterococcus hirae (ATCC 8043) bacteria were examined. Phage binding experiments were performed as described above at 4° C. and 37° C. The E. coli, Staphylococcus aureus and Enterococcus hirea strains showed equivalent binding to phage expressing peptides having the sequence of SEQ ID NO: 2 (phage 4-4-2) and SEQ ID NO: 4 (OV1 negative control), all in the range of 10⁵-10⁶ phage recovered from one ml of bacteria in mid-log phase at an O.D.₆₀₀ of 1. This level of binding is similar to the level observed with gram-negative F. philomiragia and 20-100 fold lower than the level of phage binding we see to F. novicida and F. tularensis LVS. Similar results were also obtained using Pseudomonas aeruginosa. These results show that the biopanning strategy described can identify phages expressing peptide sequences that can identify Francisella strains, such as, but not limited to, F. tularensis LVS and F. novicida and related strains, in the presence of bacteria that are routinely encountered in the clinical laboratory setting.

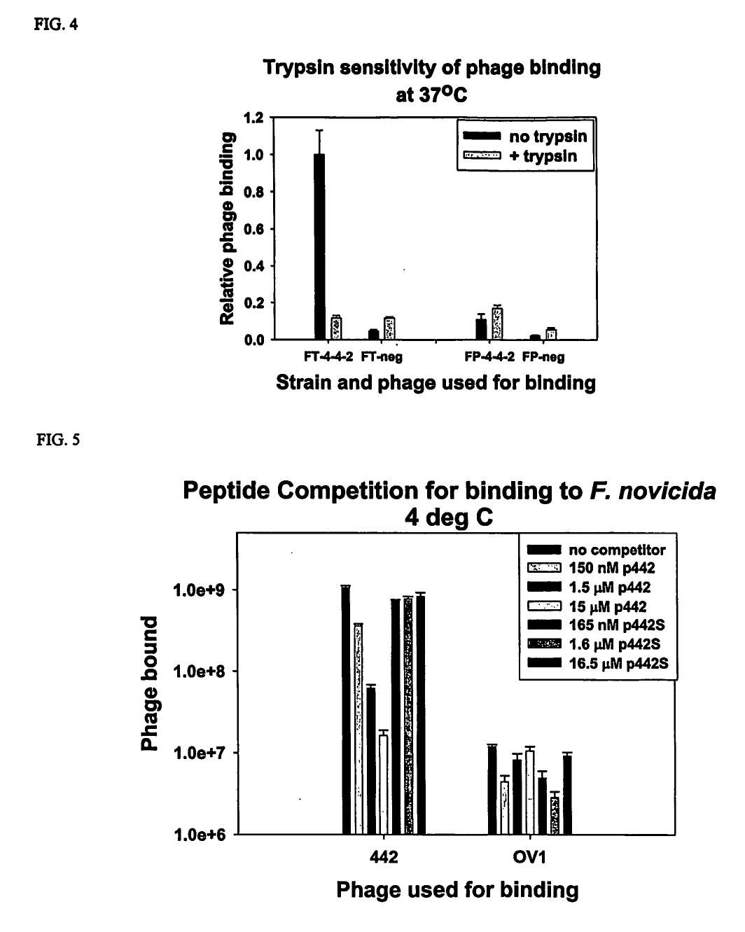

In order to characterize the target to which the peptide of SEQ ID NO: 2 binds, the surface of F. tularensis LVS and of F. philomiragia was digested with trypsin for 1 hour at 4° C. or at 37° C. (FIG. 4). Soybean trypsin inhibitor (STI) was then added to inhibit the protease and the cells were washed with 1×PBS 3 times. Phage binding experiments were performed as described above. The 37° C. experiment is shown in FIG. 4 and is typical of the results observed. For phage 4-4-2 expressing peptides having the sequence of SEQ ID NO: 2, there was a distinct trypsin sensitivity to binding to F. tularensis LVS, while there is a small but reproducible increase in binding by this phage to F. philomiragia under the same conditions. On both bacteria, the negative control phage expressing peptides having the sequence of SEQ ID NO: 4 showed substantially lower binding and an increase in binding after trypsin treatment, as if trypsin treatment reveals a target to which the phages can bind. As can be seen in FIG. 4 when compared to FIG. 2, after trypsin digestion the level of phage 4-4-2 (expressing SEQ ID NO: 2) binding to F. tularensis LVS is 10-20% that seen when no trypsin is used. This strongly suggests that the molecule to which SEQ ID NO: 2 binds is sensitive to trypsin digestion.

In order to verify that the binding observed was specific for the peptide shown in SEQ ID NO: 2, competition experiments were performed (FIG. 5.). 1×10¹¹ phage expressing peptides having the sequence of SEQ ID NO: 2 (phage 4-4-2) or SEQ ID NO: 4 (OV1 negative control) were mixed with peptides p442 having the sequence of SEQ ID NO: 2 (positive control competing peptide) or p442S having the sequence SLFWTIPIPSFR (SEQ ID NO: 5, scrambled negative control non-competing peptide having the same amino acid composition as the peptide shown in SEQ ID NO: 2). For p442, the peptide was used at concentrations of 0, 150 nM, 1.5 uM and 15 uM; for p442S, the peptide was used at concentrations of 0, 165 nM, 1.6 uM and 16.5 uM. The phage and peptide were added to 1×10⁹ F. tularensis bacteria and allowed to bind for 1 hour at 4° C. in 1×PBS. After binding, bacteria were washed three times with ice cold 1×PBS. Bound phage were eluted with 0.2 M glycine buffer (pH 2.2) and titered on E. coli strain ER2738. As can be seen from FIG. 5, the addition of p442 having the sequence of SEQ ID NO: 2 decreased binding of phage 4-4-2 to F. novicida bacteria. The addition of p442S having the sequence of SEQ ID NO: 5 (negative control) did not inhibit the binding of phage 4-4-2. Similar results were obtained for F. tularensis LVS.

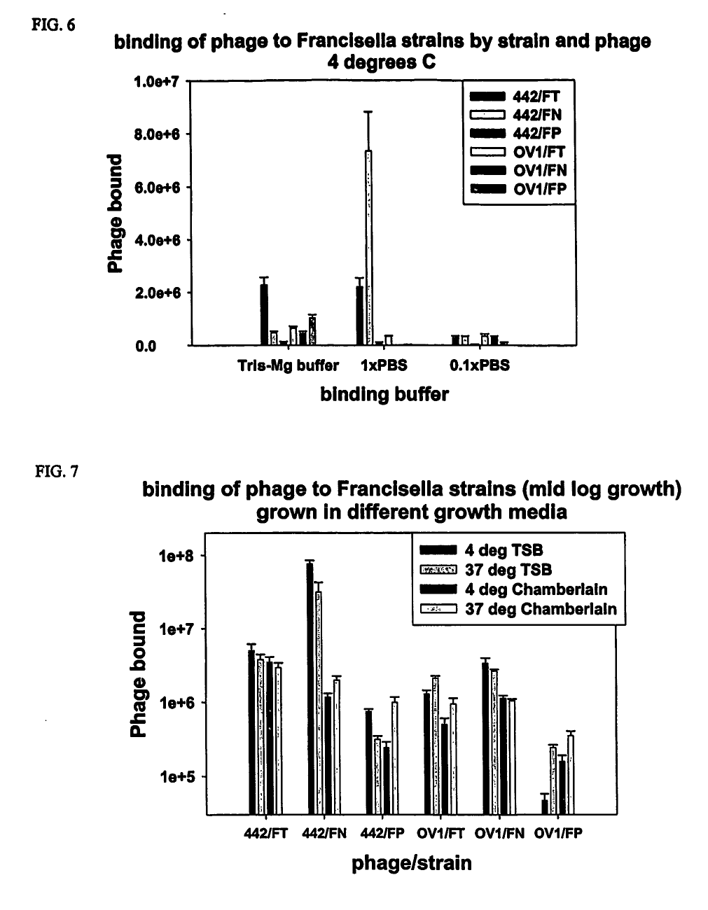

The buffers used in the binding and wash steps can also influence the binding characteristics of the reagents described in the present disclosure. The previous examples described above used 1×PBS as the binding buffer and in the various wash steps prior to phage elution. FIG. 6 shows the effects of Tris-Mg buffer, 1×PBS and 0.1×PBS on binding of phage expressing peptides having the sequence of SEQ ID NO: 2 (phage 4-4-2) or SEQ ID NO: 4 (OV1 negative control) to F. tularensis LVS, (FT), F. novicida (FN) and F. philomiragia (FP). 1×10¹¹ phage were added to 1×10⁹ bacterial cells as indicated in the various buffers shown. Phage were allowed to incubate for 1 hour at 4° C. and were washed 3 times with the same buffer used for phage binding. Bound bacteriophage were eluted with 0.2 M glycine (pH 2.2) and titered on E. coli ER2738. The number of phage bound under each condition is shown in FIG. 6. As can be seen, the use of Tris-Mg buffer favored the binding of phage 4-4-2 to F. tularensis LVS cells over F. novicida and F. philomiragia. Using Tris-Mg buffer, phage 4-4-2 showed specificity for the identification of F. tularensis LVS. Binding of the OV1 phage to F. novicida and F. Philomiragia was equal to or greater than the binding of phage 4-4-2 to these bacteria. The use of 1×PBS favored the binding of phage 4-4-2 to F. novicida over F. tularensis LVS and F. Philomiragia. However, in 1×PBS buffer, phage 4-4-2 showed specificity for the identification of F. tularensis LVS and F. novicida. Binding of the OV1 phage to F. philomiragia was equal to the binding of phage 4-4-2 for this bacterium. Of note, the number of phage 4-4-2 binding F. Tularensis LVS bacteria was not decreased significantly in the 1×PBS buffer. However, the amount of phage 4-4-2 binding to F. novicida bacteria was increased. Neither phage expressing peptides having the sequence of SEQ ID NO: 2 (phage 4-4-2) or SEQ ID NO: 4 (OV1 negative control) bound the Francisella strains tested in 0.1×PBS. This data suggests that the components of the wash and binding buffers can influence the binding specificity and selectivity with which the peptide reagents interact with their cellular targets.

The composition of the growth media used to grow the Francisella strains may also influence the binding characteristics of the reagents identified in the present disclosure. FIG. 7 shows the effect of trypticase soy broth (TSB) and Chamberlain minimal media on phage binding. TSB is a richer growth medium than Chamberlain. F. tularensis LVS AT), F. novicida (FN) and F. philomiragia (FP) bacteria were grown in either TSB or Chamberlain minimal medium to mid-log phase. Bacteria were collected by centrifugation and resuspended in 1×PBS at a concentration of 5×10⁹ bacteria/ml. 10¹¹ bacteriophage were added to the bacteria in 0.2 ml of 1×PBS at either 4° C. or 37° C. After 1 hour at 4° C. or 37° C., the bacteria were washed 3 times with 1×PBS (at the temperature used for binding). Bound bacteriophage were eluted with 0.2 M glycine (pH 2.2, at the temperature used for binding) and titered on E. coli strain ER2738. Phage used for binding were 4-4-2 (expressing peptides having the sequence of TSITPWFFLSRP) (SEQ ID NO: 2) or OV1 (expressing peptides having the sequence TGRHTPWPHFMP) (SEQ ID NO: 4) which served as a negative control. As can be seen in FIG. 7, the growth on TSB stimulated binding of phage 4-4-2 on F. novicida and F. tularensis LVS, although the stimulatory effect observed with F. tularensis LVS was not as great. This suggests that the target of the peptide expressed by the 4-4-2 phage can be increased by growth in rich media.

In order to optimize the Negative/Positive selection strategy employed in the present disclosure and to ensure the safety of the selection strategy, several assay parameters were evaluated. First, the sensitivity of Francisella strains and the phage to low pH was evaluated. The selection strategy uses a pH 2.2 wash to remove bound phage. After a 15 minute incubation of F. tularensis LVS or F. philomiragia at 4° C. with 0.2 M glycine/HCl buffer (pH 2.2), the bacteria were washed with trypticase soy broth supplemented with 0.1% cysteine (TS-C) and plated on cysteine-heart agar supplemented with 5% defibrinated horse blood (CHA-B) to assess colony formation. Treated F. philomiragia cells were unable to form colonies after incubation at low pH. Treated F. tularensis LVS cells were partially inactivated by the acidic treatment (70% inactivation). This result provides a level of biosafety to the selection strategy, since the bacteria appear to be significantly inactivated by treatment at low pH. The affect of the low pH on phage titre was also examined. 1×10⁶ phage were incubated with pH 2.2 glycine-HCl buffer for 0, 5, 10, and 20 minutes at 4° C. and at 37° C. After incubation, the phage solution was neutralized with 1 M Tris base, pH 9.1, and the phage titer determined. There was no significant decrease in the phage titer after even 20 minutes in acid indicating the phage was not adversely affected by the pH 2.2 wash.

It had been reported that heat inactivation of Francisella strains can be accomplished by incubation at 60° C. for 10-20 minutes (61). There was a dramatic decrease in viable Francisella bacteria recovered after heat treatment, with no viable colonies were recovered after 20 minutes at 60° C. In addition, incubation of M13 phages, such as, but not limited to, those found in the phage display library used in the current disclosure, at 60° C. for 1 hour does not significantly reduce the phage titer. An alternative or additional means to inactivate Francisella strains is UV inactivation (70). A UV dose of <20 J/m² will reduce the viability of F. novicida by at least 5 logs within 20 minutes at room temperature.

Independently of the pH and heat inactivation of the bacteria, filtration through a 0.22 μm filter was also shown to remove viable Francisella bacteria, as determined by plating filtered bacterial cultures on CHA-B plates and observing colony formation. As a result, the bound bacteriophages can be recovered and the Francisella bacteria eliminated in two fast and simple steps.

Finally, the ability of the added phage to lyse the Francisella strains used was evaluated. Using a spot assay with dilutions of phage lysate (10⁷, 10⁹, 10¹¹ phages per ml) on lawns of F. Tularensis LVS and of F. philomiragia, it was determined that the phages did not appear to lyse the Francisella strains used in the selection assay.

In the present disclosure, Francisella strains are grown on cysteine heart agar plates supplemented with 5% defibrinated horse blood (CHA-B) for 48 hours at 37° C., as previously described (69). For binding experiments, tryptic soybean broth supplemented with 0.1% cysteine (TS-C) was inoculated with one colony from a CHA-B plate and incubated overnight at 37° C. Inoculated broth culture are incubated until the bacteria density reaches 10⁸ to 10⁹ CFU/ml. Stock of bacteria will be stored frozen at −70° C. after addition of 10% glycerol.

Determination of Residues Critical for Binding

To further characterize the specificities involved in peptide binding, the amino acid determinants within the peptide sequence of SEQ ID NOS. 2 and 3 that are important for binding were determine using a selection method described by Sidhu and co-workers (71). In this method alanine residues are substituted at each position in a known peptide binding sequence. By careful analysis of the genetic code, this strategy can allow substitution of alanine at any position within the sequence (see Table 1, which is adapted from reference 71). After preparing such a library of alanine substituted phages with the peptide sequences of SEQ ID NOS. 2 and 3, a Negative/Positive selection was performed as described herein and bound phages recovered. Specifically, F. philomiragia was used as the surrogate strain and F. novicida was used as the target strain. A representative number of phages are sequenced and the frequency with which alanine residues appear at each position within the peptide sequence of SEQ ID NOS. 2 and 3 will be determined. Amino acids positions with a high frequency of alanine residues indicate positions not important in determining binding specificity. Positions with a low frequency of alanine residues indicate positions that are important for binding. In this manner, the critical amino acid residues responsible for binding can be determined.

Once the amino acid positions important to peptide binding are identified, these positions can be substituted to improve the binding characteristics of the peptides, such as, but not limited to, specificity and avidity, of the identified peptides using targeted selection approaches. As an alternative means to improve binding specificity and/or avidity, a new random peptide sequence library can be constructed by incorporating random amino acid sequences on or both sides of the identified peptide sequence important for binding. The number of residues added is variable and may be as few as one residue or as many as 12. With these additional peptide libraries, additional “Negative/Positive” selection as described above can be performed to identify additional peptide sequences. Additional peptide sequences can be further characterized as described above. In this manner, the specificity and/or avidity of identified peptide sequence can be modulated.

A library of alanine substituted phage sequences were produced where every position of SEQ ID NO. 2 (TSITPWFFLSRP) was substituted for alanine to determine which amino acids in SEQ ID NO. 2 are important for 44-2 phage binding. In the case of 4-4-2, this strategy results in a library approximately 260,000 different sequence permutations (6 positions with 2 amino acid choices and 6 positions with 4 possible amino acid choices). A library was prepared (final titer 10¹³ phages per ml or 3.9×10⁷ of each possible phage) and analyzed the sequences of 50 plaques to tally the number of substitutions obtained at each position (Table 2, third column). Statistically this 50 plaque sample from the library was not significantly different that predicted, except at positions S2, I3, S10, and P12; the deviation from expectation at these positions may represent sequences that provided some survival disadvantage to the phages during preparation of the library.

Five rounds of Negative/Positive selection were performed as described above with the 4-4-2 alanine-substituted phage library using F. philomiragia as the surrogate strain and F. novicida as the target strain (Table 2). After selection, the frequency was assessed for each possible amino acid found at each position within the 4-4-2 sequence. Amino acids positions with a high frequency of alanines or other amino acids after selection suggest positions that are not important in determining binding specificity of the peptide. Amino acids positions with a low frequency of alanines or other amino acids after selection suggest positions that are important in determining binding specificity of the peptide. Results from plaques picked after the Negative/Positive selection were analyzed as described (Table 2) and showed that 6 amino acids appear to be important for binding of the 4-4-2 peptide (SEQ ID NO. 2). As can be seen in Table 2, the P at position 5, the W at position 6, the F at position 7, the V at position 8, the L at position 9 and the R at position 11 are important for binding of the 4-4-2 peptide sequence (SEQ ID NO. 2). Therefore the peptide sequence important for binding is PWFVLXR (SEQ ID NO. 6) and PWFLV (SEQ ID NO. 7). In the original 4-4-2 peptide, position 8 was a phenylalanine (F), but analysis of the alanine-substituted library indicates that valine is more often recovered after selection on F. novicida (frequency 0.10 for F and 0.88 for V). The serine at position 2 and the isoleucine at position 3 also appear to be selected against Only one amino acid (the P at position 12) appears at a frequency significantly different from that expected after selection on F. Philomiragia. When the frequency is analyzed for individual peptide sequences among these 41 individual isolates after selection on F. novicida, 20 of isolated peptide sequence displayed the AATTPWFVLSRA peptide sequence (SEQ ID. NO: 8) and 12 of the isolated peptide sequences displayed the TAAAPWFVLAP peptide sequence (SEQ ID. NO: 9). In both SEQ ID NOS. 8 and 9, the core PWFVL (SEQ ID NO. 7) and PWFVLxR (SEQ ID NO. 6) sequences were retained. Specifically, the sequences PWFVLSR (SEQ ID NO. 10) and PWFVLAR (SEQ ID NO. 11) correspond to the identified conserved binding sequences.

The binding of phages AATTPWFVLSRA and TAAAPWFYLARP to F. novicida and F. Philomiragia has been tested and the affinity of binding is similar to that observed with the 4-4-2 peptide sequence (SEQ ID NO. 2).

All references cited herein are incorporated by reference to the extent allowed. The references discussed herein are provided solely for their disclosure prior to the filing date of the present application. Nothing herein is to be construed as an admission that the inventors are not entitled to antedate such disclosure by virtue of prior disclosure. The following claim is appended for the purpose of foreign priority only.

### PROPHETIC EXAMPLES

Evaluation of Binding Specificity

In order to more carefully evaluate the binding specificity of the peptides identified, fusion proteins could be constructed by incorporating one copy of the identified peptide. In the Negative/Positive selection steps discussed above, the phage expressing the peptides express 3-5 copies of each peptide. In many cases, it is desirable to characterize the binding further in a situation where only one copy of the peptide is present. For such studies, one copy of the identified peptide sequences to be tested are fused to the C-terminus of either enhanced green fluorescent protein (EGFP) or to the amino terminus of β-galactosidase or other marker protein (such as, but not limited to, but not limited to green fluorescent protein, horseradish peroxidase and other marker proteins commonly used in the art). Such constructs will allow a more carefully measurement of binding specificity, to show that binding is peptide-specific, to determine the number of binding sites, and to measure the binding constant. By using fusion proteins to the N-terminus (β-galactosidase) or C-terminus (EGFP), the importance of having a free N- or C-terminus for binding specificity or avidity can be determined.

In one embodiment, candidate peptide sequences identified by the selection strategy above will be fused to the C-terminus of EGFP. Fusion proteins will be constructed to incorporate histidine or other appropriate affinity tags for easy purification of the fusion proteins. PCR may be used to add the identified peptide sequences onto the EGFP gene in an expression plasmid. As one negative control, a histidine-tagged EGFP construct will be included. As a second set of negative controls, EGFP fusions with the same amino acid composition as the identified peptides but with the amino acids in a different random order (generated using the Genetics Computer Group program SHUFFLE) will be generated (the peptide shown in SEQ ID NO: 5 was generated using this technique). Once amplified by PCR, the DNA product will be digested with restriction endonucleases for cloning into an expression vector. In one embodiment, BglII and HindIII restriction endonucleases for cloning into the pET32a vector (Novagen) are used. Individual clones will be characterized from miniprep DNA (Qiagen) and the DNA sequence determined using the T7-reverse sequencing primer to sequence across the end of the pET32a vector. Correct clones will be transformed into an appropriate host, such as, but not limited to, E. coli BL21(DE3) cells, for expression of protein. Protein will be purified by standard methods, such as, but not limited to, by affinity purification on nickel affinity columns (Qiagen).

The purified proteins will be incubated with F. tularensis LVS or other appropriate Francisella strains to test for binding. Varying amounts of purified EGFP-peptide protein will be mixed in MH broth. After binding for 15 minutes at room temperature, at 37° C., or at 4° C., the bacteria will be washed three times with PBS (pH 7.2) or with MH broth. Binding will be assessed by the relative amount of fluorescent material visible during observation of the bacteria in a fluorescent microscope.

As an alternative, the peptide sequences can be fused to the amino terminus of β-galactosidase and X-gal used as a reagent to detect activity of the bound enzyme, by looking for the conversion of X-gal from a clear solution to a blue color. Using enzymatic methods for detection, the signal can be amplified by modulating the time for development. The fusion proteins would be constructed with histidine or other appropriate affinity tags, to allow easy purification of the fusion protein as discussed above. If necessary, any bound fusion protein can be stripped with mild acid (as done for the selection assays) or whole cells with fusion protein still bound could be used. In these experiments, an X-gal solution is incubated with the protein or cells and the OD₄₂₀ determined. The experiment will be conducted so that the reaction is in the linear range with appropriate standard curves with which to compare the observed β-galactosidase activity. Controls would include β-galactosidase fusions with peptides for scrambled sequences and β-galactosidase protein alone as discussed above.

To demonstrate binding specificity of the identified peptide, competition experiments with a competing peptide will be performed. Competing peptide would be the same sequence as the peptide attached to EGFP or β-galactosidase. For immunofluorescence experiments, varying amounts of the competing peptide would be added with known amounts of the purified EGFP-β-galactosidase-peptide fusion protein immediately prior to addition to the cells. Negative controls would be no peptide added or the scrambled peptide sequence (same amino acid composition but in a different sequence, as described above).

The binding affinity and the numbers of binding sites for the identified peptides can be determined by Scatchard Analysis as is standard in the art. In one embodiment, purified proteins will be labeled with ¹²⁵I to a specific activity of 1-2 μCi/μg, with CPM/μg determined by counting in a gamma counter and by Bradford assay. In one embodiment, the marker protein is EGFP. In this embodiment, labeled EGFP, labeled EGFP-peptide fusion proteins and labeled EGFP-scrambled peptide fusion proteins are prepared. Other marker proteins may be used, such as, but not limited to, but not limited to green fluorescent protein, horseradish peroxidase and other marker proteins commonly used in the art. The CPM/μg of protein will be obtained for each ¹²⁵I-labeled protein. F. Tularensis LVS and F. philomiragia cells, or other appropriate Francisella strains, at a known density (such as, but not limited to, 10⁸/ml) will be incubated with known amounts of labeled protein at 4° C. and at 37° C. for one hour in PBS. After binding, the bacteria will be washed with PBS and the bound and free cpm determined by counting in a gamma counter. The binding affinities and the number of binding sites will be determined using Scatchard analysis.

In addition to the above, the purified fusion proteins (EGFP, EGFP-peptide fusion proteins and EGFP-scrambled peptide fusion proteins, or their β-galactosidase counterparts) will be tested with a broad range of gram positive and gram negative bacteria to further characterize binding specificity. Examples of bacteria suitable for such use include, but are not limited to: Bacillus subtilis, Bacillus anthracis, Listeria nionocytogelzes, Staphylococcus aureus, Streptococcus pyogenes, Enterococcus hirae, Escherichia coli, Legionella pneumophila and Yersinia enterocolitica. Cultures of each of these bacteria will be incubated with varying amounts of the EGFP- or β-galactosidase-peptide fusion proteins to assess the relative binding of protein by fluorescence or by conversion of X-gal to a blue color as discussed above.

Determining the Molecule(s) to which the Identified Peptides Bind

In addition to use as diagnostic tools, the peptide reagents identified using the Negative/Positive selection strategy discussed can be used as research tools. The identity of the molecules bound by each identified peptide can be determined. The target of the identified peptides may be either a protein or other cell surface molecules, such as, but not limited to, a complex carbohydrates or lipopolysaccharides. The presence of a protein component as involved in peptide binding can be determined by trypsin digestion, or digestion with other appropriate enzymes. Using the identified peptides, it will be possible to determine what molecules are present on the target Francisella strain (for example F. tularensis LVS) that are not present on the Francisella strain used in the negative selection step. Using this approach, it will be possible to identify proteins that may be involved in the pathogenesis of Francisella strains. These cell-surface molecules might also provide research tools and specific drug targets that could be useful candidates for new therapeutics and for vaccine development. Additionally, these cell surface markers could provide new and more rapid diagnostics for tularemia.

To characterize cell surface molecules, fusion proteins expressing the identified peptide sequences or the shuffled version of the peptide sequences will be constructed. In one embodiment GST fusion proteins are used although other fusion proteins may also be employed as is known in the art. The PCR strategy to be used will be similar to that described for EGFP and -galactosidase fusion proteins above. Proteins will be expressed in a suitable host, such as, but not limited to, E. coil BL21()E3), and purified. If GST fusion proteins are used a Glutathione Sepharose 4B column (Amersham Pharmacia Biotech) can be used for purification. The same column can be used for affinity purification of the cell surface molecules, from whole cell extracts of the appropriate Francisella strain. Alternatively, the identified peptide sequences or the shuffled version of the peptide sequences can be synthesized with an N- or a C-terminal cysteine, to allow direct coupling to an affinity matrix for subsequent purification.

If the target molecule bound by the identified peptide is a protein, the target will be purified by affinity chromatography to purify it sufficiently to submit for analysis and sequencing by mass spectrometry, or other appropriate means. If the molecule is a complex carbohydrate or lipopolysaccharide, appropriate analysis of complex carbohydrates and polysaccharides will be performed.

Preparation of Cell Extracts

The target molecules to which the identified peptides bind are expected to reside on the cell surface. Whether the target molecule is a protein, LPS, complex carbohydrate or other molecule, inactivated cell extracts (prepared as described above) can be used to purify the target molecule. Cell extracts are prepared from a suitable Francisella strain. In one embodiment F. novicida is used. Because the amount of specific binding is high on F. novicida and because of the potential to perform insertional mutagenesis in this organism (69) which we will use to confirm the identity of the cell-surface molecule to which phage 4-4-2 binds, we will start purification with this organism.

Analysis of Target Molecules

To determine if the target molecule contains LPS, the methods described by Dreisbach et al (34) will be used. Appropriate Francisella cultures are collected by centrifugation, extracted with methanol and acetone, lyophilized, and then extracted with hot phenol. After treatment with DNAse and RNAse, the LPS will be analyzed for binding by the identified peptides.

To determine if the target molecule is an outer membrane protein, the methods described by Fulop et al (33) will be used. Sonication will be used to disrupt the cells and sodium N-lauroyl sarcosinate will be used to solubilize the outer membrane proteins. SDS-PAGE can be used to analyze the proteins present in this preparation. The outer membrane proteins will be analyzed for binding by the identified peptides.

Several methods may be used to analyze binding of the identified peptide to isolated proteins. The identified peptides may be labeled with ¹²⁵I, at a tyrosine residue incorporated at the end of the synthesized peptides. The purified cell protein fractions may be purified by size by SDS-PAGE, blotted with nitrocellulose or other membrane, and probed with the radioactive peptides to identify fractions containing binding activity. Alternatively, the EGFP-peptide fusions discussed above may be used to perform co-immunoprecipitation experiments to purify the bound complex in conjunction with anti-GFP antibodies. If the binding constant is not strong, it may be necessary to cross-link the proteins with the bound fusion protein complex prior to immunoprecipitation. As controls, the negative control EGFP alone and/or EGFP-scrambled peptide fusion proteins could be used. Alternatively, appropriate synthetic peptides corresponding to the peptide sequence of the fusion protein could be added to the co-immunoprecipitation reaction reduce the amount of complex recovered in a competition experiment.

The identification of the proteins identified can be carried out by a number of methods well known in the art One embodiment of an identification method is mass spectroscopy to determine amino acid sequence, followed by analysis of proteomics databases. The protein complexes may be purified by affinity chromatography or co-immunoprecipitation as discussed above. In addition, peptides extracted from 2D gels and other gels can also be analyzed. Briefly, proteins in gel pieces are hydrolyzed in situ with trypsin and the resulting peptides analyzed by matrix assisted laser desorption ionization time of flight mass spectrometry (MALDI-TOF-MS). The parent proteins are identified by submitting the profile of molecular weights of the tryptic peptides for statistical analysis using the MASCOT program at the matrixscience.com website. Confirmation of a protein's identity is carried out of the Qtof2 using MS-MS analysis. If the cell surface molecule is a protein, the protein can be identified once a mass for the protein is determined. A proteomics database containing the F. tularensis Schu-4 genome sequence is available and can be used to predict the masses of all predicted peptides in that genome.

Some of the identified peptides might recognize carbohydrate epitopes on the surface of F. Tularensis LVS. LPS will be isolated from acetone-died cells by the procedure described by Dreisbach (34) or by Vinogadov et al. (37) which involves hot phenol extraction and precipitation of proteins and nucleic acids with TCA. O-specific polysaccharide will be cleaved off the LPS by heating it in 1% acetic acid for 2.5 hr. The O-polysaccharide will then be isolated by means of gel filtration chromatography on Sephadex G-50. If peptides are found to bind to the O-specific polysaccharide, then its monosaccharide composition and structure will be further studied. Structural characterization of the O-antigen of F. tularensis strain 15 revealed that it consisted of a tetrasaccharide repeating unit containing the uncommon amino sugars, N-acetylglucosamine, N-formylquinosamine, and N-acetylgalacturonamide (37). These amino sugars formed glycosidic bonds that were resistant to the usual acid hydrolysis conditions. Despite the inability to obtain free monosaccharides upon acid hydrolysis, Vinogadov et al. (37) was able to show that strain 15 O-antigen contained a tetrasaccharide repeating unit using ¹H and ¹³C NMR procedures. Similar approaches will be used as in the analysis of O-antigen

A genetic approach may be used to confirm the identity of the cell surface marker molecule that is specifically bound by identified peptides. Based on the protein sequence obtained from the biochemical purification experiments described above, the corresponding genes will be knocked-out, and the loss of the cell surface marker on mutated bacteria will be confirmed by the absence of binding by specific peptides to F. novicida or other Francisella strain. This approach will be used when the marker has been identified as a protein and is likely to be encoded by a single gene.

The main problem with performing knock-out experiments in F. tularensis LVS is that no system to generate allelic replacement mutants have been described for this microorganism. However, F. tularensis may be used if desired. Allelic replacement has been successfully achieved in F. novicida which is very closely related to F. tularensis (69, 70). F. tularensis and F. novicida are virtually identical by 16S rDNA sequencing and by DNA hybridization and, although less virulent, F. novicida is still able to cause a tularemia-like illness in humans in rare cases (21, 22). Based on these observations, it has been suggested to consider F. novicida as a subspecies of F. Tularensis. In addition, as shown in FIG. 2, F. novicida was shown to strongly bind phage expressing SEQ ID NO: 2. Therefore, F. novicida is a legitimate surrogate for F. tularensis in our knock-out experiments.

Briefly, the presence of a target molecule identified by a peptide as described in this disclosure will be confirmed in F. novicida by Southern blot analysis. DNA probes will be identified based on the known sequence from F. tularensis Schu-4 or by creating DNA probes specific for the amino acid sequences of the purified protein product encoded by this gene. Once identified, the gene will be then isolated from F. novicida genomic DNA and cloned into an appropriate vector, such as, but not limited to, pBlueScript KMII. Either a PCR amplification strategy (if the homology between F. novicida and F. tularensis Schu4 is high in this region) or a shotgun cloning strategy followed by identification of desired clone with DNA probes) can be used for this purpose. The resulting construct will then be subjected to in vitro traposon mutagenesis, using the EZ::TN<R6Kyori KN-2> Insertion kit from Epicentre Technologies (Madison, Wis.). This kit can be used to randomly insert the <R6Kγori/KAN-2> transposon into target gene cloned into the plasmid DNA in vitro, using kanamycin as a resistance selection marker. An aliquot of the insertion reaction will then be transformed in E. coil and transformed cells will be plated on LB plates containing 50 μg/ml kanamycin. Transposon insertion clones can be sequenced from each end of the insert using forward and reverse transposon-specific primers, and only the clones carrying an insertion within the gene of interest will be selected.

The knock-out experiment will be performed as previously described (69) This approach is based on the fact that it is possible to transform F. novicida with plasmids exhibiting narrow host range (i.e., unable to replicate in F. novicida) carrying Francisella chromosomal DNA fragments and a kanwnycin resistance marker (28, 69, 70). This observation has been exploited to generate knock-out mutants by homologous recombination in F. novicida. Constructs carrying the gene of interest interrupted by EZ::TN transposon will be introduced into F. novicida by chemical transformation as previously described (70) and cells will be plated onto CHA-B plates containing kanamycin. Kanamycin-resistant transformants will be isolated after 24 to 48h incubation at 37° C. Allelic exchange between the wild-type gene and the mutated gene will be confirmed by Southern blot analysis of chromosomal DNA from each kan⁺ recombinant, using either the cloned gene itself or the EZ::Th transposon as a probe.

Mutants inactivated for a particular cell surface marker will then be tested for their binding of the corresponding peptide as described above. For example, in the case of phage 4-4-2, we expect absence of specific binding by the phage as a consequence of the loss of the marker. The binding potential of mutant F. novicida knock-outs can be determined as described above.

### REFERENCES

1. Fauci A S (2002). NIAID Strategic Plan for Biodefense Research. http://www.niaid.nih.gov/dmid/pdf/strategic.pdf.
2. Christopher G W, Cleslak T J, Pavlin J A and Eltzen E M. 1997. Biological warfare: a historical perspective. JAMA. 278: 412-417.
3. Alibek K. 1999, In Biohazard, New York N.Y.: Random House, pp. 29-38.
4. Saslaw S, Eigelsbach H T, Prior J A, Wilson H E and Carhart S. 1961. Tularemia vaccine study, II: Respiratory challenge. Arch. Intern. Med. 107: 134-146.
5. Health Aspects of Chemical and Biological Weapons. 1970. Geneva, Switzerland: World Health Organization; pp. 105-107.
6. Kaufmann A F, Meltzer M I and Schmid G P. 1997. The economic impact of a bioterrorist attack: are prevention and post-attack intervention programs justifiable? Emerg. Infect. Dis. 2:83-94.
7. Ellis J, Oyston, PCF, Green M, and Titball R W (2002). Tularemia. Clin. Microbiol. Rev. 15: 631-646.
8. Evans M E, Gregory D W, Schaffner W and McGee Z A. 1985. Tularemia: A 30-year experience with 88 cases. Medicine. 64: 251-269.
9. Brooks G F and Buchanan T M. 1970. Tularemia in the United States: Epidemiologic aspects in the 1960's and follow-up of the outbreak of tularemia in Vermont. J. Infect. Dis. 121: 357-359.
10. Olsen P F. 1975. In Hubbert W T, McCulloch W F, Schnurrenberger P R, eds. Diseases Transmitted From Animals to Man. Springfield, Ill: Charles C Thomas: 191-222.
11. Quan S F, McManus A G and von Fintel H. 1956. Infectivity of tularemia applied to intact skin and ingestion in drinking water. Science. 123: 942-943.
12. Foshay L. 1940. Tularemia: A summary of certains aspects of the disease including methods for early diagnosis and the results of serum treatment in 600 patients. Medicine. 19: 1-83.
13. Pullen R L and Stuart B M. 1945. Tularemia: Analysis of 225 cases. JAMA. 129: 495-500.
14. Tigertt W D. 1962. Soviet viable Pasteurella tularensis vaccines: A review of selected articles. Bacteriol. Rev. 26: 354-373.
15. Eigelsbach H T and Downs C M. 1961. Prophylactic effectiveness of live and killed tularemia vaccines, I: Production of vaccine and evaluation in the white mouse and guinea pig. J. Immunol. 87: 415-425.
16. Hornick R B and Eigelsbach H T. 1966. Aerogenic immunization of man with live tularemia vaccine. Bacteriol. Rev. 30: 532-538.
17. Conlan J W, KuoLee R, Shen H, and Webb A (2002). Different host defences are required to protect mice from primary systemic vs pulmonary infection with the facultative intracellular bacterial pathogen, Francisella tularensis LVS. Microbial Pathogenesis 32: 127-134.
18. Pavlova I B, Meshcheryakova I S and Emelyanova O S. 1967. Study of the microstructure of the two races of the tularemia bacterium in strains with different degrees of virulence. J. Hyg. Epidemiol. Microbiol. Immunol. 11: 320-327.
19. Hopla C E. 1974. The ecology of tularemia. Adv. Vet. Sci. Comp. Med. 18: 25-53.
20. Eigelsbach H T and McGann V G. 1986. Gram-negative aerobic rods and cocci. Genus Francisella Dorofe'ev 1947, 17^{AL}. In: Krieg N R, Hold J G, eds. Vol 1. Bergey's Manual of Systematic Bacteriology. Baltimore, Md.: Wilkins & Wilkins; 394-399.
21. Forsman M, Sandstrom G and Jaurin B. 1990. Identification of Francisella species and discrimination of type A and type B strains of F. tularensis by 16S rRNA analysis. Appl. Environmental Microbiol. 56: 949-955.
22. Horns D G, Weaver, R E, Steigerwalt A G, Wenger J D, Most C W, and Brenner D J (1989). Francisella philomiragia comb. Nov. (formerly Yersinia philomiragia) and Francisella tularensis Biogroup Novicida (formerly Francisella novicida) associated with human disease. J. Clin. Microbiol. 27:1601-1608.
23. Anthony L S D, Burke R D and Nano F E. 1991. Growth of Francisella spp. in rodent macrophages. Infect. Immun. 59: 3291-3296.
24. Fortier A H, Green S J, Polsinelli T, Jones T R, Crawford R M, Leiby D A et al. 1994. Life and death of an intracellular pathogen: Francisella tularensis and the macrophage. In Macrophage-Pathogen Interactions. Zwilling B S and Eisenstein T K eds. New York:Marcel Decker, pp. 349-361.
25. Fortier A H, Leiby D A, Narayanan R B, Asafoadjei E, Crawford R M, Nacy C A and Meltzer M S. 1995. Growth of Francisella tularensis LVS in macrophages: the acidic intracellular compartment provides essential iron required for growth. Infect. Immun. 63: 1478-1483.
26. Tärnvik A. 1989. Nature of protective immunity to Francisella tularensis. Rev. Infect. Dis. 11: 440-451.
27. Conlan J W and North R J. 1992. Early pathogenesis of infection in the liver with the facultative intracellular bacteria Listeria monocytogenes, Francisella tularensis, and Salmonella typhimurium. Infect. Immun. 60: 5164-5171.
28. Baron G S and Nano F E. 1998. MglA and MglB aro required for the intramacrophage growth of Francisella novlcida. Mol. Microbiol. 29: 247-259.
29. Holm S E, Tarnvik A, and Sandstrom G (1980). Antigenic composition of a vaccine strain of Francisella tularensis. Int. Arch. Allergy Appl. Immunol. 61:136-144.
30. Stulik J, Cerna J, Kovarova H, and Macela A (1989). Protein heterogeneity of Francisella tularensis: detection of proteins with antigenic determinants. Folia Microbiologica 34:316-323.
31. Sjostedt A, Sandstrom G, Tarnvik A, and Jaurin B (1990). Nucleotide sequence and T cell epitopes of a membrane protein of Francisella tularensis. J. Immunol. 145:311-317.
32. Sjostedt A, Sandstrom G, and Tarnvik A (1990). Several membrane polypeptides of the live vaccine strain Francisella tularensis LVS stimulate T cells from naturally infected individuals. J. Clin. Microbiol. 28:43-48.
33. Fulop M, Manchee R, and Titball R (1996). Role of two outer membrane antigens in the induction of protective immunity against Francisella tularensis strains of different virulence. FEMS Immunol. Med Microbiol 13:245-247.
34. Khlebnikov V S, Golovliov I R, Kulevatsky D P, Tokhtamysehva N V Averin S F, Zhemchugov V E, Pchelintsev S Y, Afanasiev S S, and Shcherbakov G Y (1996). Outer membranes of a lipopolysaccharide-protein complex (LPS-17 kDa protein) as chemical tularemia vaccines. FEMS Immunol. Med. Microbiol. 13:227-233.
35. Dreisbach V C, Cowley S, and Elkins K L (2000). Purified lipopolysaccharide from Francisella tularensis live vaccine strain (LVS) induces protective immunity against LVS infection that requires B cells and gamma interferon. Inf. Immun. 68:1988-1996.
36. Fulop M, Mastroeni P, Green M, and Titball R W (2001). Role of antibody to lipopolysaccharide in protection against low and high-virulence strains of Francisella tularensis. Vaccine 19:4465-4472.
37. Vinogradov E V, Shashkov A S, Knirel Y A, Kochetkov N K, Tochtamysheva N V, Averin S F, Goncharova O V, and Khlebnikov V S (199 1). Structure of the O-antigen of Francisella tularensis strain 15. Carbohyd. Res. 214: 289-297.
38. Vinogradov E, Perry M B, and Conlan J W (2002). Structural analysis of Francisella tularensis lipopolysaccharde. Eur. J. Biochem. 269: 6112-6118.
39. Conlan J W, Vinogradov E, Monteiro, M A, and Perry M B (2002). Mice internally-inoculated with intact lipopolysaccharde but not lipid A of O-chain from Francisella tularensis LVS rapidly acquire enhanced resistance against systemic and aerogenic challenge with the pathogen. Microbial Pathogenesis, in press.
40. Prior R G, Klasson L, Larsson P, Williams K, Lindler L et al. 2001. Preliminary analysis and annotation of the partial genome sequence of Francisella tularensis strain Schu 4. J. Appl. Microbiol. 91: 614-620.
41. Dennis D, Inglesby T, Henderson D, Bartlett J, Ascher M, Eltzen E, Fine A, Friedlander A, Hauer J, Layton M, Lillibridge, S F, McDade, J E, Osterholm M T, O'Toole T, Parker G, Perl T M, Russell P K, and Tonat K (2001). Tularemia as a biological weapon. JAMA 285:2763-2773.
42. Gilligan P H (2002). Therapeutic challenges posed by bacterial bioterrorism threats. Curr. Opin. Microbiol. 5:489-495.
43. Franz D R, Jahrling P B, Friedlander A M, McClain D J, Hoover D L, Bryne W R, Pavlin J A, Christopher G W, and Eltzen E M, Jr. (1997). Clinical recognition and management of patients exposed to biological warfare agents. JAMA 278:399-411.
44. Grunow R, Splettstoesser W, McDonald S, Otterbein C, O'Brien T, Morgan C, Aldrich J., Hofer E, Finke E-J, and Meyer H. (2000). Detection of Francisella tularensis in biological specimens using a capture enzyme-linked immunosorbent assay, an immunochromatographic handheld assay and a PCR. Clin. Diagnos. Lab. Immunol. 7:86-90.
45. Parmley S F and Smith G P (1988). Antibody-selectable filamentous fd phage vectors: affinity purification of target genes. Gene 73:305-318.
46. Cortese R, Monaci P, Nicosia A, Luzzago A, Felici F, Galfre G, Pessi A, Tramontano A, Sollazzo M (1995). Identification of biologically active peptides using random libraries displayed on phage. Curr. Opin. Biotechnol. 6:73-80.
47. Scott J K and Smith G P (1990). Searching for peptide ligands with an epitope library. Science 249:386-390.
48. Cwirla S E, Peteres E A, Barrett R W and Dower W J (1990). Peptides on phage: a vast library of peptides for identifying ligands. Proc. Natl. Acad. Sci. USA 87:6378-6382.
49. Felici F, Castagnoli L, Musacchio A, Jappelli R, and Cesarini G (1991). Selection of antibody ligands from a large library of oligopeptides expressed on a multivalent exposition vector. J. Mol. Biol. 222:301-310.
50. Hong S S and Boulanger P (1995). Protein ligands of the human adenovirus type 2 outer capsid identified by biopanning of a phage-displayed peptide library on separate domains of wild-type and mutant penton capsomers. EMBO J. 14:4714-4727.
51. Devlin J J, Panganiban L C and Devlin, P E (1990). Random peptide libraries: a source of specific protein binding molecules. Science 249:404-406.
52. Oldenburg K R, Loganathan D, Goldstein I J, Schultz P G and Gallop M A (1992). Peptido ligands for a sugar-binding protein isolated from a random peptide library. Proc. Natl. Acad. Sci. USA 89:5393-5397.
53. Scott J K, Loganathan D, Easley R B, Gong, X and Goldstein I J (1992). A family of concanavalin A-binding peptides from a hexapeptide epitope library. Proc. Natl. Acad. Sci USA 89:5398-5402.
54. Hoess R. Brinkmann U, Handel T. and Pastan I (1993). Identification of a peptide which binds to the carbohydrate-specific monoclonal antibody B3. Gene 128:43-49.
55. O'Neil K T, Hoess R H, Jackson S A, Ramachandran N S, Mousa S A, and DeGrado W F (1992). Identification of novel peptide antagonists for GPIIb/IIIa from a conformationally constrained phage peptide library. Proteins 14:509-515.
56. Doorbar J and Winter G. (1994). Isolation of a peptide antagonist to the thrombin receptor using phage display. J. Mol. Biol. 244:361-369.
57. Goodson R J, Doyle M V, Kaufman S E, and Rosenberg S (1994). High-affinity urokinase receptor antagonists identified with bacteriophage peptide display. Proc. Natl. Acad. Sci. USA 91:7129-7133.
58. Barry M A, Dower W J, and Johnston S A (1996). Toward cell-targeting gene therapy vectors: selection of cell-binding peptides from random peptide-presenting phage libraries. Nature Medicine 2:299-305.
59. Lee J H, Engler J A, Collawn J F, Moore B A (2001). Receptor mediated uptake of peptides that bind the human transferrin receptor. Eur. J. Biochem 268:2004-2012.
60. Cozens A L, Yezzi M J, Kunzelmann K, Ohrui T, Chin L, Eng K, Finkbeiner W E, Widdicombe J H, Gruenert D C. 1994. CFTR expression and chloride secretion in polarized immortal human bronchial epithelial cells. Am. J. Respir. Cell Mol. Biol. 10:38-47.
61. Collins F M (2002). “Pasteurella, Yersinia, and Francisella ” web site http://gsbs.utmb.edu/microbook/ch029.htm
62. Overholt E L, Tigertt W D, Kadull P J et al. 1961. An analysis of forty-two cases of laboratory acquired tularemia: Treatment with broad-spectrum antibiotics. Am. J. Med. 30: 785-806.
63. Westerman E L and McDonald J. 1983. Tularemia pneumonia mimicking Legionnaire's disease: Isolation of organisms on CYE agar and successful treatment with erythromycin. South. Med. J. 76: 1169-1171
64. Syrjälä H, Schildt R and Ralsainen S. (1991). In vitro susceptibility of Francisella tularensis to fluoroquinolones and treatment of tularemia with norfloxacin and ciprofloxacin. Eur. J. Clin. Microbiol. Infect. Dis. 10: 68-70.
65. Steichen C, Chen P, Kearney J F, and Turnbough, C L Jr. (2002). Identification of the immunodominant and other proteins of the Bacillus anthracis exosporium. J. Bacteriol., in press.
66. Knurr J, Benedict O, Heslop J, Vinson R B, McAndrew J, Kearney J F, and Turnbough C L Jr. (2002). Peptide ligands that bind selectively to spores of Bacillus subtilis and closely related species. Submitted.
67. Turnbough C L Jr. (2002). Discovery of phage-displayed peptide ligands for species-specific detection of Bacillus spores. J. Microbiol. Methods, in press.
68. Williams D D, Benedek O, and Turnbough C L Jr. (2002). Peptide ligands for the detection of Bacillus anthracis spores. Submitted.
69. Anthony S D, Gu M, Cowley S C, Leung W W S and Nano F E. (1991). Transformation and allelic replacement in Francisella spp. J. Gen. Microbiol. 137: 2697-2703.
70. Berg J M, Mdluli K E, and Nano F E (1992). Molecular cloning of the recA gene and construction of a recA strain of Francisella novicia. Infect Immun. 60:690-693.
71. Weiss G A, Watanabe C K, Zhong A., Goddard A, and Sidhu S S. (2000). Rapid mapping of protein functional epitopes by combinatorial alanine scanning. Proc. Natl. Acad. Sci. USA 97: 8950-8954.
72. Viti F, Nilsson F, Demartis S, Huber A, and Neri D (2000). Design and use of phage display libraries for the selection of antibodies and enzymes. Meth. Enzymol. 326:480-505.
73. Schaffitzel C, Hanes J, Jermutus L, and Pluckthun A (1999). Ribosome display: an in vitro method ffor selection and evolution of antibodies from libraries. J. Immunol. Meth. 231:119-135.
74. Hanes J, Schaffitzel C, Knappik A, and Pluckthun A (2000). Picomolar affinity antibodies from a fully synthetic naïve library selected and evolved by ribosome display. Nature Biotech. 18:1287-1292.
75. Wilson ds, Keefe A D, and Szostak J W (2001). The use of mRNA display to select high-affinity protein-binding peptides. Proc. Natl. Acad. Sci. USA 98:3750-3755.

76. Zaliauskiene L, Kang S, Brouillette C G, Lebowitz J, Arani R B, and Collawn J F (2000). Down-regulation of cell surface receptors is modulated by polar residues within the transmembrane domain. Mol. Biol. Cell 11:2643-265

TABLE 1
Substitutions that allow random substitution of alanine at any position
in an amino acid sequence (adapted from 71). These amino acid codons
will be initially introduced into the peptide sequence in phage plaque
4-4-2 (TSITPWFFLSRP), for selection of new peptide
sequences that might show more avidity and/or specificity for binding
to F. tularensis LVS over F. philomiragia.

| | | Possible | Expected frequency of |
|---|---|---|---|
| Amino acid | Shotgun codon | substitutions | the correct amino acid |
| F | K (C/T) T | A/F/S/V | 0.25 |
| I | (A/G) (T/C) T | A/I/T/V | 0.25 |
| L | (G/C) (T/C) T | A/L/V/P | 0.25 |
| P | (G/C) C A | A/P | 0.5 |
| R | (G/C) (G/C) T | A/R/G/P | 0.25 |
| S | (G/T) C C | A/S | 0.5 |
| T | (A/G) C T | A/T | 0.5 |
| W | (G/T) (G/C) G | A/W/G/S | 0.25 |

TABLE 2
Statistical analysis of unselected and selected 4-4-2 libraries substituted
with alanines. N = number of individual plaques whose sequences
were analyzed. P-values were assigned by analyzing the deviation of
the observed frequency for each amino acid position from the expected
frequency, using a bionomial distribution (at each position, the frequency
of the correct amino acid, with all other amino acids at that position
considered not correct). Frequencies followed by * are significantly
different from expectation (p < 0.05) and those with ** are highly
significantly different (p < 0.01). In the library selection on
F. novicida, if the data is calculated to allow V at position 8, then
the frequency at that position also becomes significantly different
from the expected frequency.

| | | Observed frequency | | |
|---|---|---|---|---|
| | | | Selected on | Selected on F. |
| | expected | Unselected | F. novicida | phihmiragia |
| position | frequency | N = 50 | N = 41 | N = 9 |
| T | 0.5 | 0.48 | 0.44 | 0.56 |
| S | 0.5 | 0.70 ** | 0.20 ** | 0.67 |
| I | 0.25 | 0.12 * | 0.00 * | 0.22 |
| T | 0.5 | 0.6 | 0.56 | 0.33 |
| P | 0.5 | 0.46 | 0.93 ** | 0.56 |
| W | 0.25 | 0.22 | 0.88 ** | 0.33 |
| F | 0.25 | 0.20 | 0.93 ** | 0.11 |
| F | 0.25 | 0.14 | 0.10 (V = 0.88 **) | 0.22 |
| L | 0.25 | 0.14 | 0.98 ** | 0.11 |
| S | 0.5 | 0.32 * | 0.54 | 0.67 |
| R | 0.25 | 0.18 | 0.85 ** | 0.22 |
| P | 0.5 | 0.74 ** | 0.49 | 1.0 ** |

1. A purified peptide comprising an amino acid sequence selected from the group consisting of SEQ ID NO: 2, SEQ ID NO: 3, SEQ ID NO: 8, and SEQ ID NO: 9.

2. The purified peptide of claim 1 the amino acid sequence is a fragment of the amino acid sequence selected from the group consisting of SEQ ID NO: 2, SEQ ID NO: 3, SEQ ID NO: 8, and SEQ ID NO: 9.

3. The purified peptide of claim 2 where the fragment comprises a contiguous sequence of at least 7 amino acids.

4. The purified peptide of claim 2 where the amino acid sequence of the fragment is selected from the group consisting of SEQ ID NO: 6, SEQ ID NO: 7, SEQ ID NO: 10, and SEQ ID NO: 11.

5. The purified peptide of claim 1 the amino acid sequence is a derivative of the amino acid sequences selected from the group consisting of SEQ ID NO: 2, SEQ ID NO: 3, SEQ ID NO: 8, and SEQ ID NO: 9.

6. The purified peptide of claim 1 where said peptide is capable of binding to a strain of Francisella.

7. The purified peptide of claim 6 where the strain of Francisella is selected from the group consisting of F. tularensis, F. tularensis LVS, F. philomiragia, and F. novicida.

8. A purified peptide comprising an amino acid sequence selected from the group consisting of SEQ ID NO: 6, SEQ ID NO: 7, SEQ ID NO: 10, and SEQ ID NO: 11.

9. The purified peptide of claim 8 where the amino acid sequence is a fragment of the amino acid sequences selected from the group consisting of SEQ ID NO: 6, SEQ ID NO: 7, SEQ ID NO: 10, and SEQ ID NO: 11.

10. The purified peptide of claim 8 where the fragment comprises a contiguous sequence of at least 3 amino acids.

11. The purified peptide of claim 6 the amino acid sequence is a derivative of the amino acid sequences selected from the group consisting of SEQ ID NO: 6, SEQ ID NO: 7, SEQ ID NO: 10, and SEQ ID NO: 11.

12. The purified peptide of claim 8 where said peptide is capable of binding to a strain of Francisella.

13. The purified peptide of claim 12 where the strain of Francisella is selected from the group consisting of F. tularensis, F. tularensis LVS, F. philomiragia, and F. novicida.

14. A purified polypeptide comprising an amino acid sequence at least 50% identical to an amino acid sequence selected from the group consisting of SEQ ID NO: 2, SEQ ID NO: 3, SEQ ID NO: 6, SEQ ID NO: 7, SEQ ID NO: 8, SEQ ID NO: 9and SEQ ID NO: 10, and SEQ ID NO: 11.

15. The purified peptide of claim 14 where said peptide is capable of binding to a strain of Francisella.

16. The purified peptide of claim 15 where the strain of Francisella is selected from the group consisting of F. tularensis, F. tularensis LVS, F. philomiragia, and F. novicida.

17-47. (canceled)

48. A diagnostic kit for detecting the presence of a Francisella strain in a sample comprising:
a. at least one peptide identified selected from the group consisting of SEQ ID NO. 2, SEQ ID NO. 3, SEQ ID NO. 6, SEQ ID NO. 7, SEQ ID NO. 8, SEQ ID NO. 9, SEQ ID NO. 10, and SEQ ID NO. 11 said peptide capable of binding to said Francisella strain; and
b. a means for detecting said peptide.

49. The kit of claim 48 where the means for detecting binding comprises detecting a detectable label that is linked to said peptide.

50. The kit of claim 49 where the detectable label is an enzyme, a chromophore, a flourophore, an affinity tag, an additional peptide sequence or a radioligand.

51. The kit of claim 49 where the detectable label is detected directly or indirectly.

52. (canceled)

53. The kit of claim 48 further comprising at least one of the components selected from the group consisting of a positive control bacterial sample, a negative control bacterial sample a negative control peptide and a set of instructions for directing the use of said kit.

54. The kit of claim 53 where the negative control peptide is a peptide having the amino acid sequence selected from the group consisting of SEQ ID NO. 4 and SEQ ID NO. 5.

55. The kit of claim 48 where the Francisella strain is pathogenic for a mammal.

56. The kit of claim 48 where the Francisella strain is selected from the group consisting of F. tularensis, F. tularensis LVS, F. philomiragia, F. novicida.

57-68. (canceled)
